(19) United States
(12) Patent Application Publication
(10) Pub. No.: US 2013/0210100 A1
(43) Pub. Date: Aug. 15, 2013

(54) ORGANOSOLV PROCESS

(71) Applicants: Alex BERLIN, Davis, CA (US); Mikhail Y. Balakshin, North Vancouver (CA); Raymond Ma, N. Vancouver (CA); Vera Maximenko Gutman, Davis, CA (US); Darwin Ortiz, Delta (CA)

(72) Inventors: Alex BERLIN, Davis, CA (US); Mikhail Y. Balakshin, North Vancouver (CA); Raymond Ma, N. Vancouver (CA); Vera Maximenko Gutman, Davis, CA (US); Darwin Ortiz, Delta (CA)

(21) Appl. No.: 13/584,697

(22) Filed: Aug. 13, 2012

Related U.S. Application Data
(63) Continuation of application No. PCT/CA2011/000183, filed on Feb. 15, 2011.
(60) Provisional application No. 61/304,755, filed on Feb. 15, 2010.
(60) Provisional application No. 61/360,377, filed on Jun. 30, 2010.

(51) Int. Cl. C12P 1/00

(52) U.S. Cl. CPC C12P 1/00
USPC 435/165; 435/267

(57) ABSTRACT

The present disclosure provides an organosolv process. The present process comprises treating a lignocellulosic biomass in the presence of a solvent and under certain conditions to separate at least a part of the lignin from the biomass.

This application is a continuation of PCT/CA2011/000183, filed Feb. 15, 2011; which claims the priority of U.S. Provisional Application Nos. 61/304,755, filed Feb. 15, 2010, and 61/360,377, filed Jun. 30, 2010. The contents of the above-identified applications are incorporated herein by reference in their entirety.

### TECHNICAL FIELD

This disclosure relates to an organosolv process. This disclosure further relates to the lignins, uses, apparatus, and the like.

### BACKGROUND

For environmental, economic, and resource security reasons, there is an increasing desire to obtain energy and material products from bio-renewable resources and particularly from “waste” and/or non-food biomass feedstocks. The various chemical components within typical biomass can be employed in a variety of ways. In particular, the cellulose and hemicellulose in plant matter may desirably be separated out and fermented into fuel grade alcohol. And the lignin component, which makes up a significant fraction of species such as trees and agricultural waste, has huge potential as a useful source of aromatic chemicals for numerous industrial applications. However, most separation techniques employed by industry today are too harsh and chemically alter the lignin component during separation to the point where it is no longer acceptable for use in many of these potential applications.

Organosolv extraction processes can be used to separate lignin and other useful materials from biomass. Such processes can be used to capitalize on the value from multiple components in the biomass. Organosolv extraction processes however typically involve extraction at higher temperatures and pressures with a volatile solvent than other industrial methods and thus are generally more complex and expensive. While large scale commercial viability had been demonstrated decades ago from a technical and operational perspective, organosolv extraction has not, to date, been widely adopted.

### SUMMARY

The present disclosure provides an organosolv process. The present process comprises treating a lignocellulosic biomass in the presence of a solvent and under certain conditions to separate at least a part of the lignin from the biomass. For example, the present process may be a biorefinery process. As used herein, the term “biorefining” refers to the co-production of bio-based products (e.g. lignin derivatives), fuel (e.g. ethanol), and energy from biomass.

As used herein, the term “organosolv” refers to bio-refinery processes wherein the biomass is subject to an extraction step using an organic solvent at an elevated temperature.

As used herein, the term “native lignin” refers to lignin in its natural state, in plant material.

As used herein, the terms “lignin derivatives” and “derivatives of native lignin” refer to lignin material extracted from lignocellulosic biomass. Usually, such material will be a mixture of chemical compounds that are generated during the extraction process.

This summary does not necessarily describe all features of the invention. Other aspects, features and advantages of the invention will be apparent to those of ordinary skill in the art upon review of the following description of specific embodiments of the invention.

### BRIEF DESCRIPTION OF THE DRAWINGS

FIG. 1 shows a typical Lignol® lignin (Alcell®) organosolv process;

FIG. 2 shows the dependence of lignin & glucose yields on solids content (L:W Ratio) at 2% acid on aspen wood, 50% EtOH in liquor, 120 min cooking time, 165° C. cooking temperature;

FIGS. 3, 4 and 5 shows the time-to-conversion target for various biomass samples.

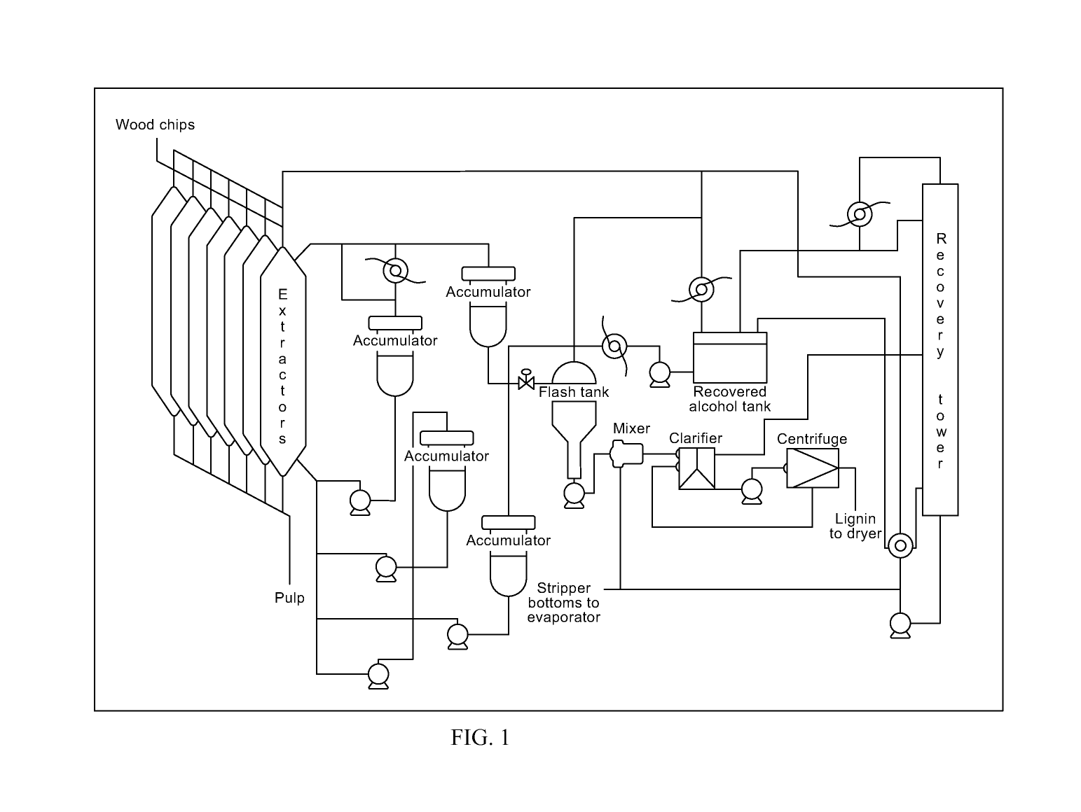

### DETAILED DESCRIPTION

The present disclosure provides an organosolv process. Organsolv processes are well known in the art. See, for example, U.S. Pat. No. 4,100,016; U.S. Pat. No. 4,764,596; U.S. Pat. No. 5,681,427; U.S. Pat. No. 7,465,791; US Patent Application 2009/0118477; US Patent Application 2009/0062516; US Patent Application 2009/00669550; or U.S. Pat. No. 7,649,086.

Four major “organosolv” pulping processes have been tested on a trial basis. The first method uses ethanol/water pulping (aka the Lignol® (Alcell®) process); the second method uses alkaline sulphite anthraquinone methanol pulping (aka the “ASAM” process); the third process uses methanol pulping followed by methanol, NaOH, and anthraquinone pulping (aka the “Organocell” process); the fourth process uses acetic acid/hydrochloric acid or formic acid pulping (aka the “Acetosolv” and “Formacell” processes).

A description of the Lignol® Alcell® process can be found, for example, in U.S. Pat. No. 4,764,596 or Kendall Pye and Jairo H. Lora, The Alcell™ Process, Tappi Journal, March 1991, pp. 113-117 (the documents are herein incorporated by reference). The process generally comprises pulping or pre-treating a fibrous biomass feedstock with primarily an ethanol/water solvent solution under conditions that include: (a) 60% ethanol/40% water (W/W), (b) a temperature of about 180° C. to about 210° C., and (c) pressure of about 20 atm to about 35 atm. Derivatives of native lignin are fractionated from the biomass into the pulping liquor which also receives solubilised hemicelluloses, other carbohydrates and other components such as resins, phytosterols, terpenes, organic acids, phenols, and tannins. Organosolv pulping liquors comprising the fractionated derivatives of native lignin and other components from the fibrous biomass feedstocks, are often called “black liquors”. The organic acid and other components released by organosolv pulping significantly acidify the black liquors to pH levels of about 5 and lower. After separation from the pre-treated lignocellulosic biomass or pulps produced during the pre-treatment process (e.g. pulping process), the derivatives of native lignin are recovered from the black liquor by flashing followed by dilution with acidified cold water and/or stillage which will cause most of the fractionated derivatives of native lignin to precipitate thereby enabling their recovery by standard solids/liquids separation processes. Various disclosures exemplified by U.S. Pat. No. No. 7,465,791 and PCT Patent Application Publication No. WO 2007/129921, describe modifications to the Lignol® Alcell® organosolv.

Organosolv processes, particularly the Lignol® Alcell® process, can be used to separate highly purified lignin derivatives and other useful materials from biomass. Such processes may therefore be used to exploit the potential value of the various components making up the biomass.

Organosolv extraction processes however typically involve extraction at higher temperatures and pressures with a volatile solvent compared to other industrial processes and thus are generally considered to be more complex and expensive. For example, when the processes are run at higher pressures (˜25-30 bar) capital costs can increase due to the necessity of using more robust equipment. In addition, the necessity of heating the biomass to high temperatures requires extra expense in terms of energy input leading to increases in operating costs.

Moreover, organosolv extraction processes can result in the production of self-precipitated lignins or lignins with poor solubility in the cooking liquor (SPLs), particularly when using softwood biomass but also when other types of biomass is used. SPLs can attach to metal surfaces causing equipment to be fouled and are difficult to remove.

In order to improve the commercial viability of organosolv processes it is desirable to keep capital and operating costs low while maximizing the potential revenue streams. For example, the cost of the enzymes used to convert the cellulose-rich pulp to mono- and/or oligosaccharides which can then be fermented into biofuels such as ethanol and n-butanol, or bio-based chemicals such as xylitol and other sugar-alcohols, succinic acid and other organic acids etc., represents a significant operating cost and, therefore, it would be advantageous to reduce the amount of enzymes needed. This may be achieved by, for example, improving the “hydrolyzability” of the pulp. Also, recovered lignin derivatives represent a source of high-value chemicals and, therefore, it would be advantageous to increase the yield of such substances. Moreover, the production of less desirable by-products, for example acetic acid, should be reduced.

Surprisingly, it has been found that organosolv processes operated within relatively narrow ranges of process conditions offer significant advantages in terms of commercial viability. For example, processes according to the present disclosure may offer improved glucose yield, lignin yield, and/or reduced production of acetic acid.

The present disclosure offers a commercially attractive organosolv process which operates at significantly lower temperature and pressure than typical for organosolv biorefining with consequent savings in capital, operating, and/or energy expenditure.

Embodiments of the present process demonstrate significantly less fouling than seen in prior art organosolv processes. For example, when the present process utilizes softwood feedstock there is a marked reduction in the amount of SPLs seen. A reduction in the amount of SPLs can result in lower equipment fouling. This offers the possibility of an improved commercial scale organosolv plant that has the ability to process softwood and other types of biomass that suffer from problems with SPLs.

Typical organosolv processes such as Lignol's® Alcell® process, generally recover around 60% of the theoretical maximum lignin. The remaining lignin is generally degraded and ends up as a waste residue. This non-recovered fraction can be toxic to microorganisms and can contaminate certain of the product streams reducing their processability by microorganisms and/or value.

Embodiments of the present disclosure offer surprisingly high lignin yields which increases the value derivable from the lignin stream of a particular process and may also reduce the amount of non-recovered lignin contaminating product streams from the process.

Embodiments of the present disclosure offer pretreated solids (“pulps”) with surprisingly good enzymatic hydrolyzability. This characteristic increases the pulps reactivity to enzymes and, hence, reduces the amount of enzyme needed for converting the pulp to sugars and subsequently to ethanol or other chemicals.

Embodiments of the present disclosure offer surprisingly high yields of glucose.

The present invention provides an organosolv process, said process comprising:

(a) pretreating (e.g. pulping) a lignocellulosic biomass with an organic solvent to form a pulp comprising cellulose and an extraction liquor comprising lignin derivatives;

(b) separating the cellulosic pulp from the extraction liquor; and

(c) recovering at least a portion of the extracted compounds from the extraction liquor.

At least a portion of the cellulosic pulp may be converted into carbohydrates, ethanol, or other chemicals.

The pretreatment step (a) of the present process can be operated at pressures of about 24 bar or less. For example, about 23 bar or less, about 22 bar or less, about 21 bar or less.

The biomass/solvent mixture of pretreatment step (a) of the present process may be heated to a temperature of from about 130° C. or greater, about 132° C. or greater, about 134° C. or greater, about 136° C. or greater, about 138° C. or greater, about 140° C. or greater, about 142° C. or greater, about 144° C. or greater, about 146° C. or greater, about 148° C. or greater, about 150° C. or greater, about 152° C. or greater, about 154° C. or greater.

The biomass/solvent mixture of pretreatment step (a) of the present process may be heated to a temperature of from about 170° C. or less, 168° C. or less, 166° C. or less, about 165° C. or less.

For example, the biomass/solvent mixture of pretreatment step (a) of the present process may be heated to a temperature of from about 155° C. to about 170° C.

The biomass/solvent mixture of pretreatment step (a) of the present process may be kept at the elevated temperature for about 45 minutes or more, about 50 minutes or more, about 55 minutes or more, about 60 minutes or more, about 65 minutes or more, about 70 minutes or more, about 75 minutes or more, about 80 minutes or more, about 95 minutes or more, about 100 minutes or more.

The biomass/solvent mixture of pretreatment step (a) of the present process may be kept at the elevated temperature for about 200 minutes or less, about 195 minutes or less, about 190 minutes or less, about 180 minutes or less, about 170 minutes or less, about 160 minutes or less, about 150 minutes or less, about 140 minutes or less, about 130 minutes or less.

For example, the biomass/solvent mixture of pretreatment step (a) of the present process may be kept at the elevated temperature for about 100 to about 140 minutes.

The solvent mixture of pretreatment step (a) of the present process may comprise about 40 wt. % or more, about 42% or more, about 44% or more, about 46% or more, about 48% or more, about 50% or more, organic solvent such as ethanol.

The solvent mixture of pretreatment step (a) of the present process may comprise about 70 wt. % or less, about 68% or less, about 66% or less, about 64% or less, about 62% or less, about 60% or less, about 58% or less, about 56% or less, organic solvent such as ethanol.

For example, the solvent mixture of pretreatment step (a) of the present process may comprise about 45 wt. % to about 60%, about 50% to about 55% organic solvent such as ethanol.

The solvent mixture of pretreatment step (a) of the present process may have a pH of from about 1.5 or greater, about 1.6 or greater, about 1.7 or greater, about 1.8 or greater, about 1.9 or greater, about 2.0 or greater, about 2.1 or greater, about 2.2 or greater, about 2.3 or greater, about 2.4 or greater, about 2.5 or greater. The solvent mixture of pretreatment step (a) of the present process may have a pH of from about 3.0 or lower, about 2.9 or lower, about 2.8 or lower, about 2.7 or lower. For example, the solvent mixture of pretreatment step (a) of the present process may have a pH of from about 2.4 to about 2.8. For example, from about 2.5 to about 2.7. For the sake of clarity, as used in this context we refer to the pH of the mixture before elevating the temperature i.e. before the ‘cook’.

From about 1.5% or greater, 1.7% or greater, 1.9% or greater, 2% or greater, by weight, of acid (based on dry weight wood) may be added to the biomass. From about 3% or lower, 2.7% or lower, 2.5% or lower, by weight, of acid (based on dry weight wood) may be added to the biomass.

The weight ratio of liquor to biomass in the pretreatment step (a) may be from about 10:1 to about 4:1, about 9:1 to about 5:1, about 8:1 to about 6:1.

The pretreatment step (a) of the present process may generate pretreated biomass solids with Time-to-Conversion-Target (TCT) equal to about 120 h or less, about 110 h or less, about 100 h or less, about 90 h or less, about 80 h or less, about 75 h or less, about 60 h or less, about 40 h or less. The pretreatment step (a) may generate pretreated biomass solids with an Overall Glucan Conversion (OGC) of about 50% or higher, about 65% or higher, about 70% or higher, about 75% or higher, about 80% or higher, about 85% or higher.

The present organic solvent may be selected from any suitable solvent. For example, aromatic alcohols such as phenol, catechol, and combinations thereof; short chain primary and secondary alcohols, such as methanol, ethanol, propanol, and combinations thereof. For example, the solvent may be a mix of ethanol & water.

The present process may utilize any suitable lignocellulosic feedstock including hardwoods, softwoods, annual fibres, energy crops, municipal waste, and combinations thereof.

Hardwood feedstocks include Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubinga; Buckeye (e.g. Aesculus hippocastanum, Aesculus glabra, Aesculus flava/Aesculus octandra); Butternut; Catalpa; Cherry (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendron ferreum, Lyonothamnus lyonii (L. floribundus), Mesua ferrea, Olea spp., Olneya tesota, Ostrya virginiana, Parrotia persica, Tabebuia serratifolia); Jacarandá; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populus×canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations/hybrids thereof.

Softwood feedstocks include Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations/hybrids thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Annual fibre feedstocks include biomass derived from annual plants, plants which complete their growth in one growing season and therefore must be planted yearly. Examples of annual fibres include: flax, cereal straw (wheat, barley, oats), sugarcane bagasse, rice straw, corn stover, corn cobs, hemp, fruit pulp, alfalfa grass, esparto grass, switchgrass, and combinations/hybrids thereof. Industrial residues like corn cobs, fruit peals, seeds, etc. may also be considered annual fibres since they are commonly derived from annual fibre biomass such as edible crops and fruits. For example, the annual fibre feedstock may be selected from wheat straw, corn stover, corn cobs, sugar cane bagasse, and combinations/hybrids thereof.

It is contemplated that any embodiment discussed in this specification can be implemented or combined with respect to any other embodiment, method, composition or aspect of the invention, and vice versa.

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. Citation of references herein is not to be construed nor considered as an admission that such references are prior art to the present invention.

Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

The present invention will be further illustrated in the following examples. However it is to be understood that these examples are for illustrative purposes only, and should not be used to limit the scope of the present invention in any manner.

### EXAMPLES

The following examples are intended to be exemplary of the invention and are not intended to be limiting.

### Example 1

All the modeling work was performed with the help of two software packages: Microsoft Excel 2007 & MatLab Version 7.7.0.471 (R2008b) with Model-Based Calibration Toolbox Version 3.5 & the CAGE Optimization Module (The MathWorks, Inc., MA, USA).

The aspen chips used for the optimization were produced by Econotech after debarking and splitting logs sourced from a local BC forest. Validation of the found optimal region was performed with aspen chips supplied by West Fraser, Alberta and screened at Lignol by Pilot Plant Operations.

Enzymatic hydrolysis was run at 50 g scale at 16% solids, 120 h, 150 rpm, pH 5.0, CellicCTec2 loaded at 12 mg/g glucan. Samples were taken after 24 h hydrolysis but here for simplicity we will report only yields after 120 h hydrolysis. This experimental design has proven to be representative of what one can see at larger scale (4-L & 20-L fermentor scale).

Thirty eight sets of five process variables (Table 1) were selected to run the optimization experiments and the results were used to build the models (Table 3).

Results & Discussion

The produced models showed that one can find pretreatment conditions for Aspen biomass where the Lignin Yield is higher than 80% and the Glucose Yield is higher than 85%. In all studied conditions the operating pressure was around 16 bar or lower.

Conclusions

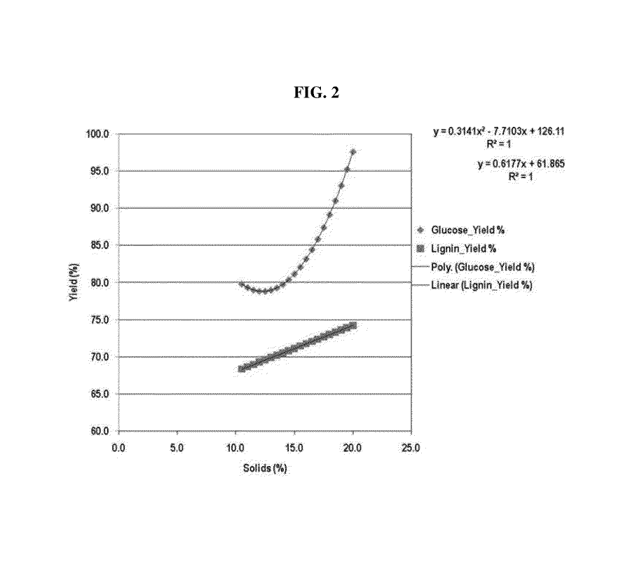

The optimum conditions for aspen lignin yield and glucose yield (˜80% or higher theoretical yields) lies between 155 and 165° C., ˜50-55% EtOH, 120-180 min cooking time, 2.0-2.5% acid at a fixed L:W ratio of 8:1 to 7:1. Any combination of these conditions yields operating pressures around or below 16 bar. Decrease of L:W ratio (increase of % solids) is beneficial and leads to increase glucose and lignin yields under certain conditions such as the ones described in FIG. 2.

TABLE 1
Sets of process variables selected to run the optimization experiments

| | Acid | Time | Temperature | Ethanol | Solids | L:W |
|---|---|---|---|---|---|---|
| # | % wt. | min | ° C. | % wt. | % | :1 |
| 1* | 2.00 | 105 | 148 | 55 | 15.0 | 5.7 |
| 2* | 2.50 | 143 | 139 | 48 | 12.5 | 7.0 |
| 3* | 1.50 | 68 | 156 | 63 | 17.5 | 4.7 |
| 4* | 1.38 | 133 | 158 | 53 | 10.6 | 8.4 |
| 5* | 1.63 | 114 | 145 | 42 | 11.9 | 7.4 |
| 6* | 2.06 | 166 | 144 | 63 | 10.3 | 8.7 |
| 7* | 1.69 | 175 | 151 | 50 | 17.2 | 4.8 |
| 8* | 2.44 | 82 | 155 | 62 | 10.9 | 8.1 |
| 9* | 2.00 | 112 | 152 | 55 | 13.1 | 6.6 |
| 10* | 1.41 | 168 | 152 | 57 | 12.7 | 6.9 |
| 11* | 2.50 | 145 | 143 | 48 | 11.6 | 7.6 |
| 12* | 2.28 | 140 | 150 | 63 | 12.0 | 7.3 |
| 13* | 1.50 | 78 | 161 | 63 | 14.6 | 5.8 |
| 14* | 1.78 | 103 | 159 | 55 | 14.5 | 5.9 |
| 15* | 2.53 | 121 | 137 | 59 | 10.8 | 8.3 |
| 16* | 1.97 | 145 | 149 | 43 | 10.5 | 8.6 |
| 17* | 2.75 | 128 | 139 | 66 | 13.9 | 6.2 |
| 18* | 1.75 | 62 | 157 | 51 | 10.9 | 8.2 |
| 19* | 1.59 | 98 | 143 | 53 | 11.1 | 8.0 |
| 20* | 1.25 | 95 | 148 | 59 | 15.4 | 5.5 |
| 21* | 1.34 | 79 | 164 | 41 | 12.3 | 7.1 |
| 22* | 2.83 | 92 | 139 | 63 | 13.8 | 6.2 |
| 23* | 2.25 | 161 | 166 | 44 | 12.4 | 7.1 |
| 24* | 1.58 | 111 | 152 | 67 | 10.1 | 8.9 |
| 25* | 2.08 | 73 | 161 | 59 | 12.6 | 7.0 |
| 26* | 1.88 | 170 | 136 | 61 | 15.0 | 5.7 |
| 27* | 1.20 | 45 | 159 | 69 | 12.0 | 7.4 |
| 28* | 2.45 | 176 | 137 | 65 | 13.2 | 6.6 |
| 29* | 2.88 | 103 | 154 | 46 | 12.0 | 7.4 |
| 30* | 1.52 | 162 | 140 | 60 | 15.4 | 5.5 |
| 31* | 2.27 | 69 | 144 | 49 | 11.6 | 7.6 |
| 32* | 2.38 | 70 | 145 | 68 | 13.5 | 6.4 |
| 33* | 2.89 | 153 | 134 | 51 | 11.0 | 8.1 |
| 34* | 1.89 | 78 | 151 | 66 | 16.0 | 5.2 |
| 35* | 2.14 | 172 | 155 | 47 | 12.3 | 7.2 |
| 36* | 1.38 | 136 | 163 | 53 | 10.5 | 8.5 |
| 37* | 1.64 | 59 | 164 | 55 | 14.8 | 5.8 |
| 38* | 2.89 | 176 | 166 | 69 | 17.5 | 4.7 |
| Min | 1.20 | 45 | 134 | 41 | 10.1 | 4.7 |
| Max | 2.89 | 176 | 166 | 69 | 17.5 | 8.9 |

TABLE 2
Output boundaries of dependent process variables

| | Lignin Yield | Glucose Yield | Washed Pulp Yield |
|---|---|---|---|
| | % | % | % bd wood |
| Min | 51.5 | 70.2 | 42.8 |
| Max | 85.1 | 87.5 | 55.8 |

TABLE 3
Lignin Yield, Glucose Yield, and Washed Pulp Yields Models

Lignin Yield (%) = −146.0292 + 27.99547 * Solids + 0.03120071 *
Temp * EtOH − 0.1121524 * Temp * Solids + 0.0015453 * Time *
Time + 0.003185002 * Time * EtOH + 0.03481116 * Time * Acid −
0.04457228 * Time * Solids − 0.03969131 * EtOH * EtOH −
0.07047815 * EtOH * Solids (r² = 0.705)
Glucose Yield (%) = −62.07805 + 2.045008 * Time − 186.731 *
Acid + 32.86724 * Solids − 0.007077812 * Temp * Time +
0.02055919 * Temp * EtOH + 0.7270062 * Temp * Acid − 0.1413414 *
Temp * Solids − 0.005416981 * Time * EtOH − 0.05171966 * Time *
Solids + 0.4617057 * EtOH * Acid − 0.2913332 * EtOH * Solids +
8.300157 * Acid * Acid + 1.758393 * Acid * Solids + 0.3140979 *
Solids * Solids (r² = 0.858)
Washed Pulp Yield (%) = −15.8281 + 0.621621 * EtOH −
5.53252 * Acid + 15.7276 * Solids + 0.00179991 * Temp * Temp −
0.0821519 * Temp * Solids + 0.00062543 * Time * Time −
0.00132139 * Time * EtOH + 0.0230202 * Time * Acid − 0.0118592 *
Time * Solids − 0.0444829 * EtOH * Solids (r² = 0.901)

### Example 2

Reactivity of Biomass Samples

“Time-to-Conversion-Target” (TCT, h) is a metric which characterizes biomass reactivity and it is defined as the time in hours required to enzymatically convert 85% of the total glucan in a pretreated biomass sample to monomeric glucose under the following reaction conditions:

12 mg protein/g glucan of the state-of-the-art enzyme CellicCTec2 (Novozymes North America Inc., Franklinton, N.C., USA). The protein content in the preparation is determined by the Pierce® Micro BCA Protein Assay Kit (Thermo Fisher Scientific Inc., Waltham, Mass., USA) in absence of interfering compounds or the enzyme protein content value is supplied by the enzyme manufacturer;

50 g total reaction weight;

16% total pretreated biomass solids in reaction;

pH 5.0, 0.1 M sodium citric buffer prepared in deionized water;

0.50 ppm antibiotic Lactrol®;

50° C.;

150 rpm mixing rate in an air incubator;

Five Zr beads per flask (Cat. No. 08-412-15C, Grinding Media for Ball Mills (Zirconia), O.D.×H 13×13 mm);

250-mL total volume of a sterilized by autoclaving glass Erlenmeyer reaction flask

The flask must be plugged with a foam plug cover by an aluminum foil to avoid evaporation or equivalent. The glucose released is measured chromatographically.

TABLE 4
Comparison of biomass reactivity (TCT, h) between three differently pretreated
aspen samples

| Biomass | Pₘₐₓ | Acid | | Time | | EtOH | Solids | L:W | TCT |
|---|---|---|---|---|---|---|---|---|---|
| Sample | (bar) | (% wt) | pH | (min) | T (° C.) | (% wt) | (% wt) | Ratio | (h) |
| S10005865 | 9.7 | 1.20 | 2.20 | 45 | 159 | 69 | 12 | 7.40 | 168.6 |
| 28(1) | | | | | | | | | |
| 14(1) | 12.8 | 1.50 | 2.02 | 78 | 161 | 63 | 15 | 5.85 | 121.5 |
| S10005636 | 11.0 | 1.80 | 1.90 | 103 | 159 | 55 | 15 | 5.90 | 117.5 |
| 15(1) | | | | | | | | | |

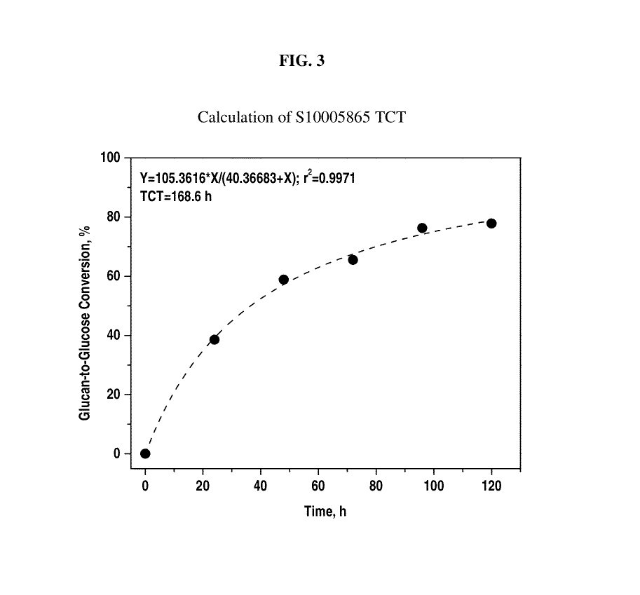

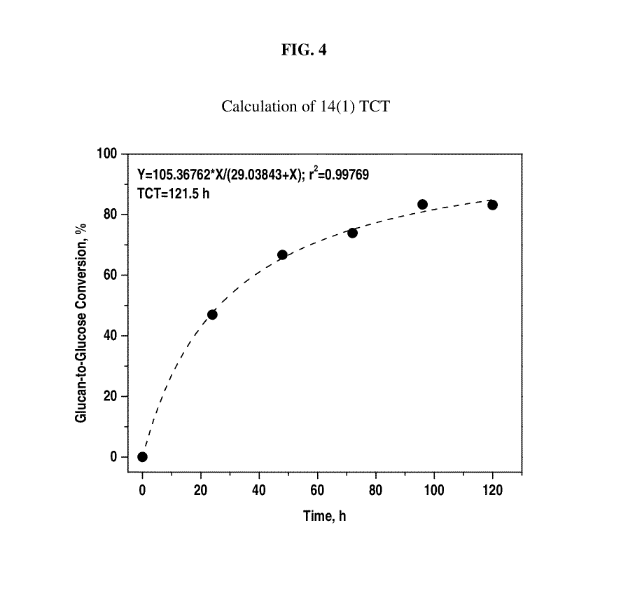

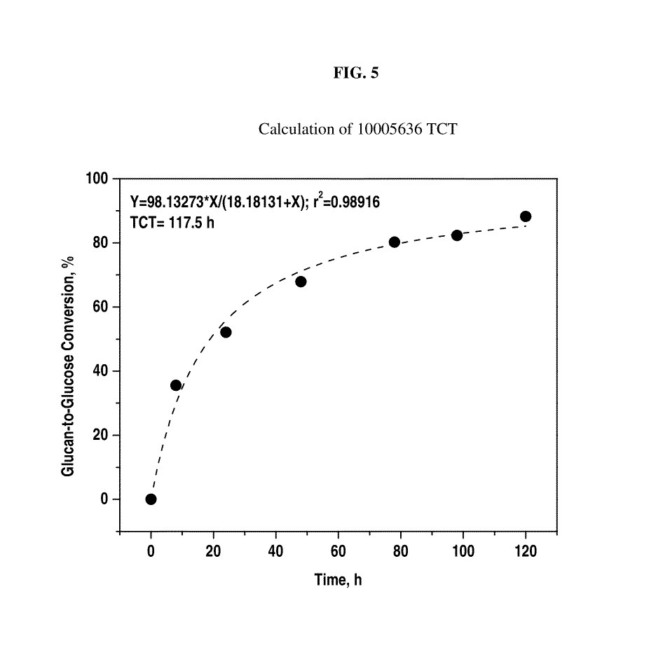

The pretreated biomass sample S10005636 15(1) shows the highest reactivity with the shortest time (117.5 h) required to achieve the target (85% glucan-to-glucose conversion) while the sample S10005865 28(1) shows the lowest reactivity with a 168.6 h TCT. The TCT values are calculated by extra- or intrapolation using the experimental hyperbolic functions Glucan-to-Glucose Conversion (%) vs. Time (h) (FIGS. 3-5). These hyperbolic functions are typical of enzymatic hydrolysis reactions.

“Overall Glucose Conversion” (OGC, % total glucose in raw biomass) is a metric which provides the total glucose recovered from the pretreated solids in fermentable monomeric form and it integrates both the glucose recovery yield after biomass pretreatment (PGY—Pretreatment Glucose Yield) and the glucose hydrolysis yield after enzymatic hydrolysis (HGY—Hydrolysis Glucose Yield). The OGC is calculated as follows:

OGC (%)=Recovered_Glucose_After_Pretreatment_per_{—}100 g Pretreated_Raw_Material (g)*HGY(%)

TABLE 5
Overall Glucose Conversion (OGC, % total glucose in raw biomass)
yields of three differently pretreated aspen samples

| Biomass | Pₘₐₓ | Acid | | Time | | EtOH | Solids | L:W | OGC |
|---|---|---|---|---|---|---|---|---|---|
| Sample | (bar) | (% wt) | pH | (min) | T (° C.) | (% wt) | (% wt) | Ratio | (%) |
| S10005865 | 9.7 | 1.20 | 2.20 | 45 | 159 | 69 | 12 | 7.40 | 78.17 |
| 28(1) | | | | | | | | | |
| 14(1) | 12.8 | 1.50 | 2.02 | 78 | 161 | 63 | 15 | 5.85 | 76.57 |
| S10005636 | 11.0 | 1.80 | 1.90 | 103 | 159 | 55 | 15 | 5.90 | 79.11 |
| 15(1) | | | | | | | | | |

“Maximum Operating Pressure” (Pₘₐₓ, bar) is defined as the maximum operating pressure reached during the biomass pretreatment stage. In the case of the present invention this value is around 16 bar or lower.

“Best Pretreated Biomass” (BPB) is defined as the pretreated biomass produced under Pₘₐₓ around or lower than 16 bar which shows the lowest TCT and the highest OGC with the highest lignin yield. The lignin yield must be considered for economic reasons but it does not necessarily impacts biomass reactivity.

In the case of the three compared pretreated aspen samples the BPB is the sample S10005636 15(1) since it showed the highest OGC and the lowest TCT while the maximum operating pressure (11 bar) was kept well below the allowed maximum of 16 bar.

### Example 3

Various types of feedstock were processed in the pilot plant facility at Lignol Innovations Inc, Burnaby, Canada. Numerous runs were performed (Table 6). The resultant pulp glucan to ethanol conversion was 80% or greater. In addition, no significant issues were observed with SPLs.

TABLE 6
Pilot Plant Cooking Conditions/Results

| | | | | | | | | Best attained |
|---|---|---|---|---|---|---|---|---|
| | | | | | | | | Pulp |
| | | | | Primary | Acid | | | Glucan- |
| | | | | Ext. | Loading | | | Ethanol |
| | Feed- | No. of | | Time | (% OD | Temp. | Pressure | Conversion |
| Ext. | stock | cooks | L/W | (min) | wood) | (° C.) | (kPa) | (%) |
| A | Aspen | 43 | 7-10 | 120 | 2-2.5 | 165 | 2100 | 81 |
| | Alder | 51 | 7-10 | 120 | 2-2.5 | 165 | 2100 | 81 |
| | BKLP* | 83 | 7-10 | 120 | 2.5-3.5 | 165 | 2100 | 80 |
| B | Aspen | 181 | 7-8 | 120 | 2-2.5 | 165 | 2100 | 81 |
| | Alder | 13 | 7-8 | 120 | 2-2.5 | 165 | 2100 | 81 |
| | BKLP* | 13 | 7.5-10 | 120 | 2.5-3.5 | 165 | 2100 | 80 |

*Beetle Killed Logpole Pine

1. An organosolv process for treating a lignocellulosic biomass comprising:
a. Adding the biomass to a reaction vessel and exposing the biomass to a solvent wherein:
i. the ratio of solvent to biomass is from about 10:1 to about 4:1;
ii. the solvent comprises from 40 to 60% w/w ethanol;
iii. from about 1.5% to about 2.5% (based on dry weight wood) of an acid is added;
b. Elevating the temperature of the biomass/solvent mixture to from about 140° C. to about 170° C. for a total period of from about 50 minutes to about 200 minutes to form a pulp and an extraction liquor said liquor comprising extracted derivatives of native lignin;
c. Recovering at least a portion of the extraction liquor;
d. Recovering at least a portion of the pulp; and
wherein the pressure in the reaction vessel is less about 22 bar.

2. An organosolv process for treating a lignocellulosic biomass comprising:
a. Adding the biomass to a reaction vessel and exposing the biomass to a solvent wherein:
i. the ratio of solvent to biomass is from about 10:1 to about 4:1;
ii. the solvent comprises from 40 to 60% w/w ethanol;
iii. the pH of the biomass/solvent mixture is from about 2.5 to about 2.7;
b. Elevating the temperature of the biomass/solvent mixture to from about 150° C. to about 170° C. for a total period of from about 50 minutes to about 200 minutes to form a pulp and an extraction liquor said liquor comprising extracted derivatives of native lignin;
c. Recovering the extraction liquor;
d. Recovering the pulp; and
wherein the pressure in the reaction vessel is about 22 bar or less.

3. The process of claim 1 or 2 wherein the pressure in the reaction vessel is about 21 bar or less.

4. The process of claim 1 or 2 wherein the elevated temperature of step (b) is from about 160° C. to about 170° C.

5. The process of claim 1 or 2 wherein the elevated temperature of step (b) is maintained for about 100 minutes to about 140 minutes.

6. The process of claim 1 or 2 wherein the solvent comprises from 50% to 60% w/w ethanol.

7. The process of claim 1 or 2 wherein from about 2% to about 2.5% (based on dry weight wood) of an acid is added.

8. The process of claim 1 or 2 wherein the ratio of solvent to biomass is from about 7:1 to about 5:1.

9. The process of claim 1 or 2 wherein the biomass comprises softwood feedstock.

10. The process of claim 1 or 2 wherein the biomass comprises hardwood feedstock.

11. The process of claim 1 or 2 wherein the biomass comprises annual fibre feedstock.

12. The process of claim 1 or 2 wherein the yield of lignin recovered from the extraction liquor is 60% or greater of the theoretical maximum yield.

13. The process of claim 1 or 2 wherein the yield of lignin recovered from the extraction liquor is 70% or greater of the theoretical maximum yield.

14. The process of claim 1 or 2 wherein the yield of lignin recovered from the extraction liquor is 80% or greater of the theoretical maximum yield.

15. The process of claim 1 or 2 wherein the pretreated biomass solids have a TCT equal to 120 h or less and an OGC of 50% or higher.

16. The process of claim 1 or 2 wherein at least a portion of the recovered pulp is converted into carbohydrates which are subsequently converted into ethanol.
